(19) United States
(12) Patent Application Publication
(10) Pub. No.: US 2021/0007976 A1
(43) Pub. Date: Jan. 14, 2021

(54) Segmented EVA Intravaginal Rings

(71) Applicant: Robert Prince, Poway, CA (US)

(72) Inventors: Bridget Martell, Guilford, CT (US); David Friend, San Diego, CA (US); Martin Doorbar, Hampshire (GB); Shen Luk, Nottingham (GB)

(21) Appl. No.: 16/835,251

(22) Filed: Mar. 30, 2020

Related U.S. Application Data
(60) Provisional application No. 62/826,978, filed on Mar. 29, 2019.
(60) Provisional application No. 62/843,288, filed on May 3, 2019.

(51) Int. Cl. A61K 9/00; A61K 47/32; A61K 31/565; A61K 31/57

(52) U.S. Cl. CPC A61K 9/0036; A61K 31/57; A61K 31/565; A61K 47/32

(57) ABSTRACT

Disclosed herein are segmented, EVA intravaginal rings that release 17β-estradiol and progesterone with specific pharmacokinetics useful for treating, ameliorating, and preventing symptoms associated with menopause and vulvar and vaginal atrophy.

### RELATED APPLICATION

This application claims the benefit of priority to U.S. Ser. No. 62/826,978, filed Mar. 29, 2019 and U.S. Ser. No. 62/843,288, filed May 3, 2019. The disclosures of which are incorporated by reference herein, including drawings.

### FIELD OF THE INVENTION

The present invention is in the field of intravaginal rings, in particular segmented, ethylene-vinyl acetate (EVA) intravaginal rings, and their use treating, ameliorating, or preventing, vasomotor symptoms in perimenopause and/or menopausal women and for treating, ameliorating, or preventing vulvar and vaginal atrophy (VVA).

### BACKGROUND OF THE DISCLOSURE

During menopause, there is a decline in ovarian function, with the precipitous fall of circulating levels of estrogen leading to physiological changes and the associated symptoms of menopause. While the causal relationship between low estradiol E₂ levels and genitourinary symptoms is well understood,¹ the mechanism behind vasomotor symptoms (VMS) is less well elucidated, but it is clear that E₂, although not solely responsible, is a major endocrine influencer.²

The recently published position statement of the North American Menopause Society states that hormonal therapy (HT) is the most effective treatment for VMS and genitourinary symptoms, with both local and systemic treatments widely used. Hormonal therapy with low dose vaginal estrogen therapy is recommended for isolated genitourinary symptoms associated with vulvovaginal atrophy (VVA). Treatment with E₂ alone or in combination with a progestogen is recommended as the first-line therapy for VMS in women without contraindications, where the addition of a progestogen is needed to provide endometrial protection from unopposed systemic estrogen.³⁻⁵ The use of Progesterone (P) is preferred by some over progestins in patients at risk of cardiovascular disease, due to favorable effects on lipids, atherosclerosis, and vascular reactivity.^{6,7}

There are currently two E₂-only silicone-based intravaginal rings (IVRs) approved in the United States (US) for the treatment of menopausal women: a 17β-estradiol acetate vaginal ring (Femring: equivalent to release of 50 μg/day and 100 μg/day) approved to treat VMS and local VVA symptoms and an E₂ vaginal ring (Estring: 7.5 μg/day) approved only for local vaginal symptoms. A combination product that delivers E₂ and P via an IVR over a 28-day period, which treats women for VMS and VVA, a known urogenital symptom of menopause, while providing endometrial protection, would be novel and desirable. However, there are currently no approved nonoral combination products in the US that contain both E₂ for treatment of VMS and VVA and Progesterone, necessary for endometrial protection.

The coadministration of E₂ and P in the same IVR that delivers a consistent and reliable non-oral dose over a 28-day period would be a valuable option for the treatment of VMS and urogenital symptoms of menopause.

### SUMMARY OF THE INVENTION

Disclosed herein are ethylene-vinyl-acetate (EVA), intravaginal rings (IVRs), wherein the rings contain at least two segments/fibers, wherein one segment contains 17β-estradiol (E₂) or E₂ equivalent and the second segment contains progesterone (P) or P equivalent.

In some embodiments the ring is about 57 mm in diameter with a cross-section diameter of about 5 mm.

In some embodiments, the EVA rings release about 80 μg/day E₂ to about 160 μg/day E₂.

In some embodiments, the EVA rings release about 80 μg/day E₂ or about 160 μg/day E₂.

In some embodiments, the EVA rings release about 4 mg to about 8 mg P per day.

In some embodiments, the EVA ring segment/fiber containing E₂ is prepared at a concentration of about 10 w/w in EVA (28% vinyl acetate content).

In other embodiments, the EVA segment/fiber length containing E₂ is about 15 mm long.

In still other embodiments, the EVA ring segment/fiber containing P is prepared using EVA (28% vinyl acetate content) with a final drug loading of 27% w/w.

In yet other embodiments, the EVA segment/fiber length is about 74.5 mm or about 148.5 mm in length.

In some embodiments, the IVR rings have a peak E₂ plasma concentration at about 4 hours after insertion for the IVR rings releasing about 4 mg/day P and about 2 hours after insertion for the EVA rings releasing about 8 mg/day P.

In other embodiments, the disclosed EVA rings result in a mean E₂ AUC₀₋₆₇₂ₕᵣ value of about 17,400±2,120 pg*hr/mL for rings releasing 4 mg/day P.

In some embodiments, the EVA rings have a mean E₂ AUC₀₋₆₇₂ₕᵣ value of about 21,000±3,540 pg*hr/mL for EVA rings releasing about 8 mg/day P.

In some embodiments, the EVA rings disclosed herein the average plasma concentration over the entire dosing interval (C_{AVG}) is about 25.9±3.16 pg/mL for rings releasing 4 mg/day P and about 31.3±5.26 pg/mL for rings releasing about 8 mg/day P.

In some embodiments, the EVA rings have in vivo release for rings releasing about 4 mg/day P that is about 55.9±6.8 μg/day and about 67.3±11.3 μg/day for rings releasing about 8 mg/day P.

In other embodiments, the EVA rings disclosed herein result in an E₂ plasma concentration of about 130 pg/ml-180 pg/ml.

In some embodiments, the EVA rings disclosed herein result in Cₘₐₓ values for rings releasing 4 mg/day P of about 1,590±272 pg/mL and about 2,400±322 pg/mL for rings releasing about 8 mg/day of P.

In some embodiments, the EVA rings have a C_{AVG}, with mean values of about 357±11.2 pg/mL for rings releasing about 4 mg/day P, and about 722±94.1 pg/mL for rings releasing about 8 mg/day P.

In still other embodiments, the EVA rings maintain E₂ concentrations at a quantifiable level at day 29 following IVR insertion into a patient/subject.

In some embodiments, the disclosed EVA rings has one of more of the pharmacokinetic properties found in Table 1-4 and FIGS. 2-4.

In some embodiments, disclosed herein are methods of treating, ameliorating, or preventing vasomotor symptoms associated with perimenopause or menopause in a patient/subject in need of treatment using the disclosed EVA rings.

In some embodiments, the symptoms treated, ameliorated, or prevented by the disclosed EVA rings are selected from one or more of hot flashes, hot flushing, night sweating, mood swings, anxiety, urinary incontinence, cognitive disturbances (memory loss, problems with concentration, arthralgia, weight gain, sexual dysfunction, vaginal disturbances, sleep disturbances, such as insomnia, bone loss, heart disease, atherosclerosis, and heart palpitations.

In some embodiments, the disclosed EVA rings treat, ameliorate, or prevent the symptoms associated with vulvar and vaginal atrophy (VVA) in a patient/subject in need of treatment using an EVA ring disclosed herein.

In some embodiments, the disclosed EVA rings, treat, ameliorate, or prevent symptoms of VVA symptoms selected from one or more of dryness, burning, itching, vaginal discomfort, vaginal discharge, pain and burning when urinating, urgency with urination, increased urinary tract infections, urinary incontinence, dyspareunia, discomfort with intercourse, decreased vaginal lubrication during sexual activity, shortening and tightening of the vaginal canal, and spotting during intercourse.

### BRIEF DESCRIPTION OF THE DRAWINGS

FIG. 1. Schematic showing process used to prepare E₂ and P releasing EVA IVRs.

FIG. 2. In vitro release profiles of E₂ (Panel A) and P (Panel B) from IVRs (160/4) or 8 [160/8]. Values are mean values (n=6)±SD.

FIG. 3. Plasma concentration-time profiles of E₂ following a single intravaginal insertion of comparator IVRs Femring (50 or 100 μg/d E₂) or the 160/4 and 160/8 IVRs in female sheep. Data are means (n=5)±SD intravaginal ring.

FIG. 4. Plasma concentration-time profiles of P following a single intravaginal insertion of the 160/4 or 160/8 IVRs in female sheep. Data are means (n=5)±SD intravaginal ring.

### DETAILED DESCRIPTION OF THE EMBODIMENTS

Disclosed herein are IVRs that allow for E₂ and P to be integrated into a single ethylene-vinyl acetate (EVA) ring-delivery system. The IVRs disclosed herein can be used to treat (e.g., cure, suppress), ameliorate, and/or prevent (e.g., delaying or preventing die onset, recurrence or relapse of) one or more symptoms of perimenopause or menopause, such as without limitation VMS in women with an intact uterus, while also aiming to treat, ameliorate, an/or prevent the symptoms of VVA. VMS symptoms can include, without limitation, one or more of the following: hot flashes, flushing, night sweating, mood swings, anxiety, urinary incontinence, cognitive disturbances (memory loss, problems with concentration, arthralgia, weight gain, sexual dysfunction, vaginal disturbances, sleep disturbances, such as insomnia, bone loss, heart disease, atherosclerosis, and heart palpitations. VVA symptoms can include, without limitation, one or more of dryness, burning, itching, vaginal discomfort, vaginal discharge, pain and burning when urinating, urgency with urination, increased urinary tract infections, urinary incontinence, dyspareunia, discomfort with intercourse, decreased vaginal lubrication during sexual activity, shortening and tightening of the vaginal canal, and spotting during intercourse.

The disclosed IVRs are designed to either deliver about 80 to about 160 μg/day E₂ with an adequate dose of P for endometrial protection over a 28-day period. Previous reports using IVRs comprised of an inner core of silicone elastomer matrix containing E₂ and P, and a silicone elastomer overcoat, have demonstrated the effectiveness of delivering 160 μg/day E₂ with either 10 or 20 mg/day P in 20 postmenopausal women.⁸ Results showed a significant reduction in the incidence of hot flashes and night sweats from 2 weeks through 16 weeks, with ultrasound monitoring of the endometrium suggesting effective protection against endometrial hyperplasia.

In some embodiments of the invention described herein, where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. For example, a range from 27% to 36% would include 27% to 29%, or 27% to 33% or 33% to 35%, etc. Such ranges would also include individual points in the range, for example, 28%, 29%, 30% etc.

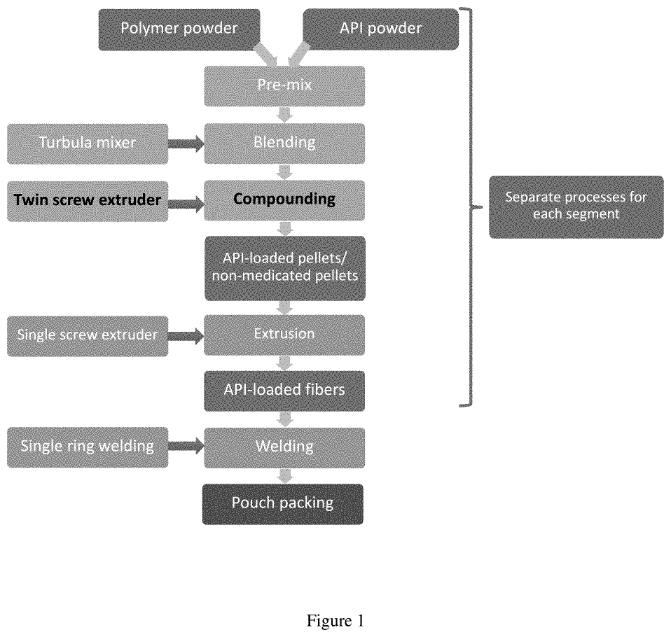

The disclosed IVRs are made by a process described in FIG. 1 and in a manner similar to that described previously.¹³ The process comprises compounding pellets, extrusion of fibers followed by joining of the fibers by heat welding. Blending can be accomplished using a Turbula mixer (Model T 10 B, with a 17-liter stainless steel mixing vessel, Glenn Mills, Clifton, N.J.). The resulting blends were then compounded by hot-melt extrusion using a twin-screw extruder (Pharma 11 Twin Screw Hot Melt Extruder with a Pharma 11 gravimetric feeder) and fed onto a Pharma 11 Air Cooled conveyor followed by pelletization using a Pharma 11 Vericut Pelletizor (Thermo Fisher Scientific, Dreieich, Germany). The pellets were formed into fibers by hot melt extrusion using a 25 mm single screw extruder (Dr Collin, Ebersberg, Germany). The resulting fibers were cut using a Dr Collin in-line Cutting Station. Cut fibers (or segments) were welded using Automationspartner single station laboratory welder (Ramlosa, Sweden).

IVRs capable of releasing E₂ (EP, Aspen Oss B.V., Oss, The Netherlands) at the desired rates were prepared by using fibers of varying length and drug loading. In some embodiments, the IVRs described herein release E₂ at rate pf about 160 μg/d and P released at about 4 mg/d (160/4 IVR) or about 8 mg/d (160/8 IVR). In some embodiments, the described IVRs are about 57 mm in overall diameter with a cross sectional diameter of about 5 mm.

The EVA fiber containing E₂ was prepared at a concentration of about 10 w/w in EVA (28% vinyl acetate content, Vitaldose®, Celanese Corporation, Boucherville, Canada). The E₂ fiber length in the finished IVR is about 15 mm in both the 160/4 and 160/8 IVRs. IVRs releasing about 4 and about 8 mg/d P were prepared using EVA (28% vinyl acetate content, Vitaldose) with a final drug loading of about 27% w/w. To create the 160/4 IVR, the P-containing segment length was about 74.5 mm with a placebo segment length of about 74 mm. To create the 160/8 IVR, 27% loaded EVA fiber was about 148.5 mm in length. There was no placebo segment in the 160/8 IVR.

As used herein “about” means approximately plus or minus 10% of the indicated value.

In an aspect disclosed herein, IVR rings made according the process described in FIG. 1 and as described herein are used to treat VMS and/or VVA symptoms. In some embodiments of this aspect, the IVR rings contain one or more segments containing E₂. In some embodiments, the IVR rings contain one or more segments containing P. In some embodiments, the IVR rings contains both P and E₂ each in a separate segment or segments.

In some aspects, the disclosed IVRs are about 57 mm in overall diameter with a cross sectional diameter of about 5 mm.

In some aspects the EVA fiber (segment) containing E2 is prepared at a concentration of about 10 w/w in EVA (28% vinyl acetate content, Vitaldose®, Celanese Corporation, Boucherville, Canada or Polysciences, Inc., Warrington, Pa.) or equivalent from other manufacturers of vinyl acetate. In some embodiments, the E₂ fiber length in the finished IVR is about 15 mm.

In some aspects, IVRs releasing about 4 mg and about 8 mg/d P are prepared using EVA (28% vinyl acetate content, Vitaldose) with a final drug loading of about 27% w/w. In some embodiments, the P-containing segment length is about 74.5 mm. In some embodiments, the P-containing segment length is about 148.5 mm in length.

In some aspects, the disclosed IVR rings have a peak E₂ plasma concentration at about 4 hours for the 160/4 IVR after insertion and about 2 hours after insertion for the 160/8 IVR.

In some embodiments, the maximun observed E₂ plasma concentrations (Cmax) values are about 149±21.3 pg/mL for the 160/4 IVR and about 158±54.6 pg/mL for the 160/8 IVR.

In some embodiments, the mean E₂ AUC₀₋₆₇₂ₕᵣ value for the 160/4 ring is about 17,400±2,120 pg hr/mL,

In some embodiments, the mean E₂ AUC₀₋₆₇₂ₕᵣ value for the 160/8 IVR is about 21,000±3,540 pg hr/mL.

In some embodiments, the average plasma concentration over the entire dosing interval (C_{AVG}) is about 25.9±3.16 pg/mL for the 160/4 IVR and about 31.3±5.26 pg/mL for the 160/8 ring.

In some embodiments, the in vivo release for the 160/4 IVRs and the 160/8 IVRs E₂ is about 55.9±6.8 μg/day and 67.3±11.3 μg/day, respectively.

In some embodiments, the disclosed 160/4 or 160/8 IVRs, the median peak P plasma concentrations is observed 4 hours after insertion for both IVRs [4 mg/day (160/4) and 8 mg/day (160/8)].

In some embodiments, the mean P Cₘₐₓ and AUC₀₋₆₇₂ₕᵣ values increase with increasing dose for the disclosed IVRs. In some embodiments, the Cₘₐₓ values for Groups 3 (160/4) and 4 (160/8) were about 1,590±272 pg/mL and about 2,400±322 pg/mL, respectively. In some embodiments, there is a 2-fold increase in AUC₀₋₆₇₂ₕᵣ values (240,000±7,510 pg*hr/mL (160/4) and 485,000±63,200 pg*hr/mL (160/8). In some embodiments, the disclosed rings have C_{AVG}, with mean values of 357±11.2 pg/mL in Group 3 (160/4) and 722±94.1 pg/mL in Group 4b (160/8).

In some embodiments, the disclosed IVR rings maintain E₂ concentrations at a quantifiable level at Day 29 following IVR insertion in all animals

In some embodiments, the disclosed rings can effectively release E₂ and/or P for a period of about 7 days or more, or a period of about 10 days or more, or a period of about 14 days or more, or a period of about 20 days or more, or for a period of about 26 days, or for a period of about 27 days, or for a period for about 28 days, or for a period for about 29 days, or for a period of about 30 days, or for a period up to 30 days, or for a period up to 29 days, or for a period up to 28 days.

The studies described herein were conducted in vivo in sheep, a relevant nonclinical model to determine the pharmacokinetics (PK) and local tolerability of E₂ and P combination IVRs compared with a marketed 17β-estradiol acetate IVR Femring® (Warner Chilcott, Rockaway, N.J.), that releases the equivalent of E₂ at either 50 μg/day or 100 μg/day. A sheep model was selected because the vagina and cervix in sheep are reported to be of a size similar to those in women,⁹ thereby allowing for the testing of IVRs without modification of ring dimensions. Sheep are routinely used in the nonclinical assessment of intravaginal compounds and IVRs^{9,10} as the vagina is composed of a stratified squamous epithelium that is more comparable to humans than other commonly used models of intravaginal administration (e.g., rabbits). Lastly, the response of the sheep vagina to endogenous and exogenous estrogenic and progestogenic stimulation is well described.^{11,12}

### In Vitro Release of E₂ and P

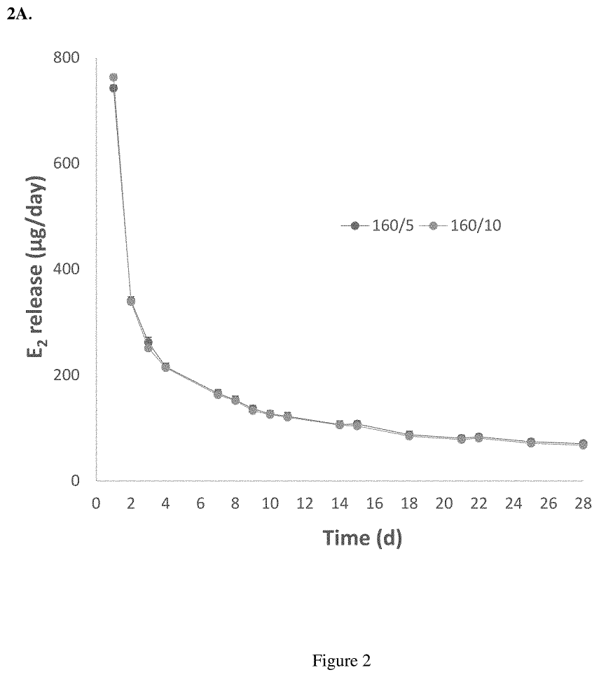

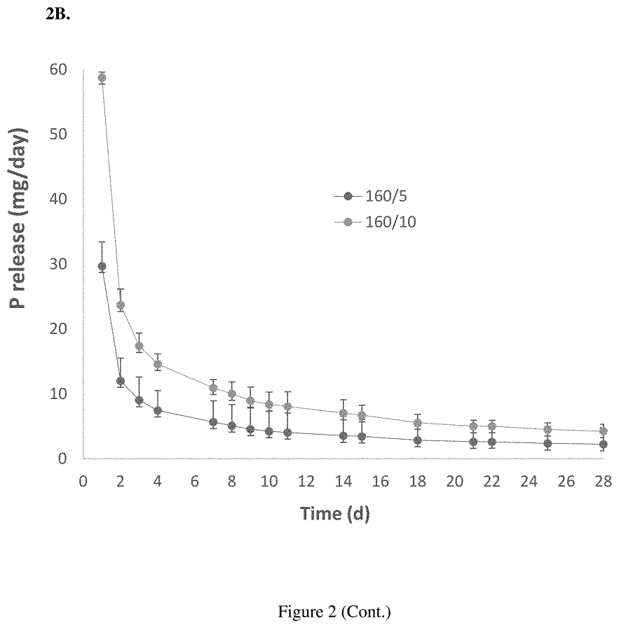

Release rates of E₂ and P over the 28-day test period were characterized by the rate over Day 1, from Day 2 to Day 28, and the rate on Day 28. Table 1 shows the data collected in this manner from the different 160/4 and 160/8 IVRs. The in vitro release profiles of both drugs are typical of a matrix-type delivery system with a relatively rapid release of drug followed by a period of slower release, which can be seen in FIG. 2.

TABLE 1
Release rates of E₂ and P from the 160/4 and 160/8 IVRs

| Test Time | | 160/4 IVR | | 160/8 IVR | |
|---|---|---|---|---|---|
| Points | | E₂ | P | E₂ | P |
| 0-24 | h | 742 ± 6.2^{a} | 30 ± 3.7 | 762 ± 5.6 | 58.7 ± 0.8 |
| 2-28 | d | 122 ± 6.9 | 4.1 ± 2.7 | 119 ± 3.0 | 7.9 ± 0.9 |
| 28 | d | 70.3 ± 5.5 | 2.2 ± 2.6 | 67.3 ± 2.8 | 4.2 ± 1.1 |

^{a}All data are means ± SD (n = 6)

### Pharmacokinetics

Following removal of the IVRs on Day 29, analysis of the residual E₂ and P showed that all rings were within ±10% of the theoretical mass balance of both hormones, with the exception of the ring obtained from the Group 4 animal that was released on Day 18.

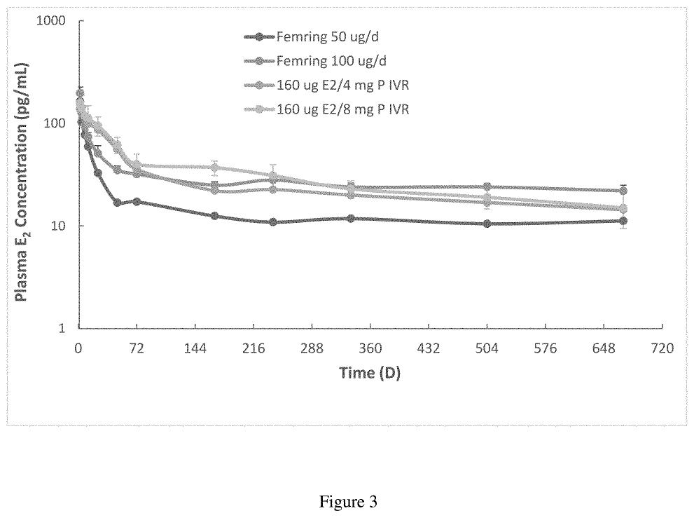

The plasma concentrations of E₂ from the two comparator IVRs (Femring, 50 μg/d and 100 μg/d) and from the 160/4 and 160/8 IVRs are shown in FIG. 3. PK parameters of E₂ from these four IVRs groups over the 28-day release period are presented in Table 2. Peak E₂ plasma concentrations following 160/4 and 160/8 IVR insertion on Day 1 were observed at a median of 4 hours in Group 3 and at 2 hours after insertion in Group 4, respectively. Maximum observed E₂ plasma concentrations (Cₘₐₓ) values were 149±21.3 pg/mL and 158±54.6 pg/mL in Groups 3 and 4, respectively. E₂ concentrations remained at a quantifiable level at Day 29 following IVR insertion in all animals. The mean E₂ AUC₀₋₆₇₂ₕᵣ value in Group 3 was 17,400±2,120 pg*hr/mL, lower than that observed in Group 4 (21,000±3,540 pg*hr/mL). The average plasma concentration over the entire dosing interval (C_{AVG}) was also numerically lower in Group 3 than in Group 4 (25.9±3.16 pg/mL and 31.3±5.26 pg/mL, respectively).

TABLE 2
E₂ PK parameters from intravaginal rings

| | | Group 1 | | Group 2 | | Group 3 | | Group 4 | |
|---|---|---|---|---|---|---|---|---|---|
| | | Treatment | | | | | | | |
| | | 50 μg/d | | 100 μg/d | | | | | |
| | | comparator ring | | comparator ring | | 160/4 IVR | | 160/8 IVR | |
| | | Mean | | Mean | | Mean | | Mean | |
| PK Parameter | | (SD) | CV % | (SD) | CV % | (SD) | CV % | (SD) | CV % |
| Cₘₐₓ | (pg/mL)^{a} | 164 (26.8) | 16.3 | 197 (29.6) | 15.0 | 149 (21.3) | 14.3 | 158 (54.6) | 34.6 |
| AUC_{0-672 hr} | (pg*hr/mL)^{b} | 9,690 (1,750) | 18.0 | 19,000 (1,170) | 6.19 | 17,400 (2,120) | 12.2 | 21,000 (3,540) | 16.8 |
| C_{AVG} | (pg/mL)^{c} | 14.4 (2.6) | 18.0 | 28.2 (1.75) | 6.19 | 25.9 (3.16) | 12.2 | 31.3 (5.26) | 16.8 |
| R₀ | (μg/day)^{d} | 31.0 (5.6) | 18.1 | 60.8 (3.8) | 6.25 | 55.9 (6.8) | 12.2 | 67.3 (11.3) | 16.8 |

^{a}Cₘₐₓ, maximum observed plasma concentration
^{b}AUC_{0-672 hr}, area under the plasma concentration-time curve from 0 to 672 hours
^{c}C_{AVG}, average plasma concentration over the entire dosing interval
^{d}R₀, calculated in vivo release rate

Animals administered comparator IVRs (Femring 50 μg/d or 100 μg/d E₂ release rates) had peak E₂ plasma concentrations observed at 2 hours after IVR insertion on Day 1 for both comparator ring groups. The mean Cₘₐₓ values were comparable between the 2 dose groups; despite the 2-fold difference in theoretical E₂ release rates, there was only a 1.2-fold increase in Cₘₐₓ between Group 1 (164±26.8 pg/mL) and Group 2 (197±29.6 pg/mL). In contrast, the AUC₀₋₆₇₂ₕᵣ values increased proportionally with increasing dose; a 2-fold increase in dose resulted in a 2-fold increase in AUC₀₋₆₇₂ₕᵣ values, from 9,690±1,750 pg*hr/mL in Group 1 to 19,000±1,170 pg*hr/mL in Group 2. Similarly, the average plasma concentration over the entire dosing interval (C_{AVG}) saw a 2-fold increase from 14.4±2.6 pg/mL in Group 1 to 28.2±1.75 pg/mL in Group 2.

The calculated in vivo release rate (R₀) can be determined using the PK data and the equation R₀=C_{AVG}×CL, where C_{AVG} is obtained individually from each animal in Groups 3 and 4 and CL is the total clearance obtained with intravenous administration (Table 4). From 160/4 IVRs (Group 3) and the 160/8 IVRs (Group 4) E₂ in vivo release rates in this sheep model were 55.9±6.8 μg/day and 67.3±11.3 μg/day, respectively. These estradiol values bracket the value obtained from the Group 2 comparator administration, at 60.8±3.8 μg/day.

Across the different IVRs tested (the comparator silicone Femring IVRs versus EVA-derived 160/4 and 160/8 IVRs), the release rates and hence AUCs for E₂ differed, with the release rates being lower for the 160/4 and 160/8 IVRs despite higher E₂ concentrations being loaded into the EVA matrix. This lower release is presumed due to a different IVR construct that contributes to the release characteristics of the IVR (e.g. EVA vs. silicone for the comparator).

For the animals receiving the 160/4 or 160/8 IVRs, the median peak P plasma concentrations were observed 4 hours after insertion for both dose groups (4 mg/day and 8 mg/day). P was quantifiable up to 21 days (2 animals) or 28 days (3 animals) after IVR insertion in Group 3 and up to 28 days following IVR insertion in Group 4.

Mean P Cₘₐₓ and AUC₀₋₆₇₂ₕᵣ values increased with increasing dose (Table 3). The Cₘₐₓ values in Groups 3 and 4 were 1,590±272 pg/mL and 2,400±322 pg/mL, respectively. There was a 2-fold increase in AUC₀₋₆₇₂ₕᵣ values (240,000±7,510 pg*hr/mL and 485,000±63,200 pg*hr/mL, respectively). A 2-fold increase was also then observed between dose groups for C_{AVG}, with mean values of 357±11.2 pg/mL in Group 3 and 722±94.1 pg/mL in Group 4.

TABLE 3
P pharmacokinetic parameters from 160/4 and 160/8 IVRs

| | | Group 3 | | Group 4 | |
|---|---|---|---|---|---|
| | | Treatment | | | |
| | | 160/4 IVRs | | 160/8 IVRs | |
| | | P dose (mg/day) | | | |
| | | 4 | | 8 | |
| PK Parameter | | Mean (SD) | CV % | Mean (SD) | CV % |
| Cₘₐₓ | (pg/mL) | 1,590 (272) | 17.1 | 2,400 (322) | 13.4 |
| AUC_{0-672 hr} | (pg*hr/mL) | 240,000 (7,510) | 3.13 | 485,000 (63,200) | 13.0 |
| C_{AVG} | (pg/mL) | 357 (11.2) | 3.13 | 722 (94.1) | 13.0 |
| R₀ | (μg/day) | 3523 (112) | 3.18 | 7132 (923) | 12.9 |

Group 5 (n=3) received a single intravenous bolus administration of 160 μg E₂ and 10 mg P. Data from 1 animal were considered aberrant, due to an unexplained second peak in E₂ and P concentrations 3 hours postdose, and were excluded. In the remaining animals, the mean E₂ AUC_{Inf} was 1,790 pg*hr/mL; for P, the mean AUC_{Inf} was 20,000 pg*hr/mL (Table 4).

The in vivo release rates from the PK data were similarly calculated for P from the 2 IVRs as described above for E₂. The in vivo release rates were 3.5 and 7.1 mg/day for Groups 3 and 4, respectively, similar to the labeled release rates of 4 and 8 mg/day.

TABLE 4
E₂ and P PK parameters following a
single intravenous administration

Group 5
Treatment

| | |
|---|---|
| E₂ (n = 2^{a}) | P (n = 2) |

Intravenous Dose

| | 160 μg | 10 mg |
|---|---|---|
| PK Parameter | Mean | Mean |

| | | | |
|---|---|---|---|
| AUC_{INF} | (pg*h/mL)^{a} | 1,790^{a} | 20,000^{a} |
| CL | (L/hr)^{b} | 89.6^{b} | 500^{b} |
| V_{z} | (L)^{c} | 287^{c} | 1,980^{c} |
| T_{1/2} | (h)^{d} | 2.22^{d} | 2.75^{d} |

^{a}AUC_{INF}, area under the plasma concentration-time curve from time zero extrapolated to infinity
^{b}CL, total body clearance
^{c}V_{z}, volume of distribution based on terminal elimination phase
^{d}T_{1/2}, terminal half-life
^{e}Standard deviations and coefficients of variation were not calculated since n = 2

### Animals—Observations

Observations collected during the treatment period were unremarkable for abnormal physical findings. Enlarged udders were observed in several animals (including some animals with enlargement prior to administration of study treatment), but since all animals had a history of lactation, these observations were not considered to be related to the study treatment. A clear vaginal discharge was observed in 1 animal from Group 4, but discharge findings are common during studies with the intravaginal route. Red discoloration of the vulva was observed in some animals across all treatment groups. Mean body weight was increased by approximately 10% relative to 1 week prior to treatment in all groups (1 to 4) on Day 29.

Following administration of the IVRs, daily vaginal ring checks confirmed that the IVRs remained in place until the time of removal (Day 29) in all but 1 animal. For 1 animal in Group 4, the ring was found to have been expelled on Day 16. Following a visual inspection for damage, the ring was washed and reinserted, but was expelled again on Day 18 and could not be located. A new ring was inserted and remained in place until Day 29. Blood samples from this animal were not used in the PK analyses.

### In-Life Vaginal Irritation Scoring

Evaluations of external vaginal irritation performed on Days 1 through 29 of the treatment period comprised assessments of erythema and edema on a scale of 0 to 4 (Table 5). The number of assessments showing well-defined erythema and eschar was low and comparable across the 4 treatment groups. Erythema developed on Day 2 in all animals and was most severe at the outset of treatment. Very slight erythema (scores of 1) persisted longer in Groups 3 and 4 than in Groups 1 and 2; the incidence of observations of very slight erythema/eschar was greatest in the Group 4 animals, with most animals experiencing very slight erythema for the majority of the study period (data not shown).

Evaluation of the external vagina for edema showed no scores greater than 1 (very slight edema) in any animal. The incidence of very slight edema was greatest in Group 2, the high-dose comparator ring group. In both of the 160/4 and 160/8 IVR groups, the incidence of very slight edema was comparable to that in Group 1. Internal vaginal assessment on Day 29 after removal of IVRs showed little or no irritation in both comparator ring groups. The incidence of observations of erythema was greater in the 160/4 and 160/8 IVR groups than in the comparator ring groups and was higher in the 8 mg/day progesterone group than the 4 mg/day dose (Table 5).

### Postmortem Assessments:

Postmortem macroscopic assessment of the vagina showed treatment-related changes in the 160/4 and 160/8 IVR-treated animals; red foci were observed in the cranial vagina, near the cervix, of those animals who had erythema in the Day 29 in-life internal examinations (2 in Group 3, and 5 in Group 4).

Microscopic observation showed changes in the 160/4 and 160/8 IVR-treated animals, which were more frequent in the cranial vagina (Table 6). The changes observed showed a degree of correlation with the P dose. In the cranial vagina, focal areas showing minimal atrophy were observed in 2 animals in Group 3, and 4 animals in Group 4; these areas were characterized by focal areas of thinning epithelium, with minimal mononuclear cell infiltration in the underlying tissue. Ulceration was observed in the cranial vagina of 1 animal in each of the 160/4 and 160/8 IVR groups (1 minimal, 1 mild). For both animals, infiltration of monocytes and neutrophils was reported, the extent of which correlated with the severity of the ulceration. Areas of atrophy and ulceration generally correlated with the red foci that were observed macroscopically.

Epithelial hyperplasia was observed at all regions of the vagina in Groups 1, 2, and 3 and considered minimal to mild (Tables 6 and 7). The incidence and severity of hyperplasia were lower in the Group 3 (160/4 IVR animals) than in the comparator ring groups, with no epithelial hyperplasia observed in the Group 4 (160/8 IVR animals) at any region of the vagina. Similarly, within the cervix, squamous metaplasia was observed in all treatment groups, but was more frequent and more severe in the comparator ring groups than the 160/4 or 160/8 IVR groups, consistent with unopposed estrogen treatment. As is expected, all (ovariectomized) groups treated with exogenous estradiol treatment had minimal uterine hypertrophy when compared to ovariectomized control animals. These changes would be consistent with the general stimulation associated estrogenic treatment in ovariectomized animals (data not shown).

### EXAMPLES

### Intravaginal Rings

Intravaginal rings capable of releasing E₂ and P were prepared in a manner similar to that described previously.¹³ The overall process is shown schematically in FIG. 1. The process involved compounding pellets, extrusion of fibers followed by joining of the fibers by heat welding. Blending was accomplished using a Turbula mixer (Model T 10 B, with a 17-liter stainless steel mixing vessel, Glenn Mills, Clifton, N.J.). The resulting blends were then compounded by hot-melt extrusion using a twin-screw extruder (Pharma 11 Twin Screw Hot Melt Extruder with a Pharma 11 gravimetric feeder) and fed onto a Pharma 11 Air Cooled conveyor followed by pelletization using a Pharma 11 Vericut Pelletizor (Thermo Fisher Scientific, Dreieich, Germany). The pellets were formed into fibers by hot melt extrusion using a 25 mm single screw extruder (Dr Collin, Ebersberg, Germany). The resulting fibers were cut using a Dr Collins in-line Cutting Station. Cut fibers (or segments) were welded using Automationspartner single station laboratory welder (Ramlösa, Sweden). Manufacturing of IVRs was performed by QPharma, Malmö, Sweden.

IVRs capable of releasing E₂ (EP, Aspen Oss B.V., Oss, The Netherlands) at the desired rates are prepared by using fibers of varying length and drug loading. The two IVRs evaluated release E₂ at rate pf 160 μg/d and P released at 4 mg/d (160/4 IVR) or 8 mg/d (160/8 IVR). All IVRs are 57 mm in overall diameter and a cross sectional diameter of 5 mm. The EVA fiber containing E₂ was prepared at a concentration of 10 w/w in EVA (28% vinyl acetate content, Vitaldose®, Celanese Corporation, Boucherville, Canada). The E₂ fiber length in the finished IVR is 15 mm in both the 160/4 and 160/8 IVRs. IVRs releasing 4 and 8 mg/d P were prepared using EVA (28% vinyl acetate content, Vitaldose) with a final drug loading of 27% w/w. To create the 160/4 IVR, the P-containing segment length was 74.5 mm with a placebo segment length of 74 mm. To create the 160/8 IVR, 27% loaded EVA fiber was 148.5 mm in length. There was no placebo segment in the 160/8 IVR.

In Vitro Release of E₂ and P from IVRs

The release rates of E₂ and P from the 160/4 and 160/8 IVR formulations were measured in vitro to determine whether the target release rates had been attained. Release rates were tested using 200 mL 0.5% sodium dodecyl sulfate as a release medium, in shakers operating at 130±2 rpm at 37° C. Sampling (2 mL) was conducted at 6 h, days 1-4, 7-11, 14, 15, 18, 21, 22, 25, and 28. Concentrations of E₂ and P were determined using a validated reverse-phase liquid chromatography method. The column used was a Phenomenex Luna C8(2), 150 mm×3.0 mm, 5 μm and the guard column was Phenomenex C8 (4 mm×3 mm). The mobile phase was acetonitrile 45% in purified water (55%), v/v. The injection volume was 10 μL. Detection of E₂ was based on florescence (279 nm excitation with 306 nm emission); P was detected by UV at 245 nm. The standard curve range for E₂ was 0.25-3.5 μg/mL; the range for P was 0.00625-0.25 mg/mL. Both curves were linear (correlation coefficient>0.997). Six IVRs were tested at each dissolution time point.

### Animal Study Design

The purpose of this study was to evaluate the in vivo PK and local tolerability of 160/4 and 160/8 IVRs in drug-naïve ovariectomized female Dorset crossbred sheep and to establish the comparative bioavailability with 50 or 100 μg/day E₂ comparator IVR products (Femring) over a 28-day exposure period, and with a single IV administration of a 160 μg E₂ and 10 mg of P.

The study was conducted at MPI Research, A Charles River Co., which is an American Association for Accreditation of Laboratory Animal Care (AAALAC) accredited contract research organization. It was conducted in compliance with the US Food and Drug Administration (FDA) Good Laboratory Practices (GLP) Regulations and the US Department of Agriculture (USDA) Animal Welfare Act.

A total of 27 experimentally naïve, female, uniparous, Dorset crossbred sheep, approximately 15.5 to 19 months of age at receipt, were received from Ehrhardt Farm, Eaton Rapids, Mich. and from Lauwers Lamb, Capac, Mich. Animals were identified by implanted microchips and by individual ear tags.

During acclimation, the animals were observed daily with respect to general health and any signs of disease. All animals were given a detailed examination, and body weights were recorded within 3 days of receipt and again prior to the operating procedures. All animals were negative for Cryptosporidium and Giardia species. Strongyloides and Coccidia were detected in stool samples from almost all animals. Animals were treated with a single administration of fenbendazole (10 mg/kg orally). Animals weighed 57.5 to 77.0 kg at randomization.

Between 25 and 54 days before the scheduled dosing, all animals underwent a surgical procedure to remove the ovaries, in accordance with the research facility's standard operating procedures.

Animals were allowed to recover for 25 to 54 days prior to dosing. During this recovery period, body weight measurements and other observations were performed weekly. Ovariectomy surgery was performed successfully in all animals as determined by undetectable levels of endogenous hormones.

During the course of the study, all animals were observed twice daily for morbidity, mortality, injury, and the availability of food and water. Detailed examination of each animal was performed weekly during the study. These observations included evaluation of the skin, fur, eyes, ears, nose, oral cavity, thorax, abdomen, external genitalia, limbs and feet, respiratory and circulatory effects, autonomic effects such as salivation, and nervous system effects including tremors, convulsions, reactivity to handling, and unusual behavior.

Animals were randomly allocated to 1 of 5 treatment groups: Group 1 (n=5) comparator IVR Femring (50 μg E₂/day); Group 2 (n=5) comparator IVR Femring (100 μg E₂/day); Group 3 (n=5) 160/4 IVR; Group 4 (n=5) 160/8 IVR; and Group 5 (n=3) bolus injection of 160 μg E₂ and 10 mg P.

The 160/4 and 160/8 IVRs were stored at 2 to 8° C. until use and were allowed to warm to room temperature for 30 to 120 minutes prior to use; comparator IVRs were stored at room temperature in accordance with labeling. All IVRs were inserted on Day 1 and were to remain in place until removal at Day 29. Vaginal ring insertion was performed as a clean procedure. The IVR was photographed before being digitally inserted into the cranial vagina using a gloved finger. During the treatment period, animals were digitally examined daily to confirm that the IVR was still in place.

Following completion of treatment on Day 29, the IVRs were removed from each animal and were photographed and stored at 2 to 8° C. before being returned for analysis of residual E₂ and P content. The theoretical mass balance was calculated by adding the amount of E₂ and P present in the device after use with the amount released by the IVR (taken from the initial release testing of the IVRs in vitro) and dividing by the theoretical drug content. This was calculated as a gross check on IVR performance, and results were not intended to be correlated with PK findings.

E₂ and P were administered to Group 5 on Day 1 via 2 separate intravenous injections into the jugular vein, at a dose volume of 1 mL/dose. P was administered first, followed within 1 to 3 minutes by E₂. The control articles were used as received from the supplier (Sigma Aldrich, Milwaukee, Wis.), formulated to achieve nominal concentrations of 0.16 mg/mL E₂ or 10 mg/mL P in ethanol/propylene glycol/sterile water (3:3:4, v/v/v), and filtered through a 0.22 μm polyvinylidene fluoride syringe filter prior to administration.

### Pharmacokinetics

Blood samples taken periodically from 2-672 hours were placed in tubes containing K₂-EDTA and were centrifuged under refrigerated conditions within 60 minutes of sample collection. The resulting plasma was stored frozen at −60 to −90° C. within 120 minutes of sample collection. Plasma samples were shipped on dry ice for analysis (Pyxant Labs, Inc., Colorado Springs, Colo.). Plasma samples were analyzed using liquid chromatography-mass spectrometry/mass spectrometry methods validated according to bioanalytical method guidelines. The standard curve range for E₂ was 5-500 pg/mL; the range for P was 0.1-20 ng/mL. Based on quality control samples, accuracy ranged from 96.7-101% for E₂ and 96.5-98.0% for P. Precision (% CV) was less then 4.2% for E₂ and less than 7.5% for P. The lower limit of quantitation (LLOQ) for E₂ was 5.0 pg/mL and 0.1 ng/mL for P; the upper limit of quantitation for E₂ was 100 pg/mL and 20 ng/mL for P. Concentrations below the LLOQ were set to zero for PK analyses.

Standard noncompartmental PK analysis methods were used. Pharmacokinetic parameters were determined for E₂ and P (as applicable). The area under the concentration-time curve (AUC) values were estimated by the trapezoidal rule, and intravenous clearance was estimated as dose/AUC_{INF}. The C_{avg} following IVR administration was calculated as AUC₀₋₆₇₂ₕᵣ/672.

### Tolerability: Description of Assessments, Grading Scale

For animals in Groups 1 through 4, examination of the external vagina (the vulva and the externally visible portion of the vestibule) was conducted before insertion of the IVRs, and daily examinations were conducted on Days 2 through 29, prior to the daily ring checks. The external vagina was observed for gross signs of irritation (i.e., erythema and edema) and any other signs of local or systemic effect. Irritation was scored based on the Draize scale¹⁴; erythema and edema formation were rated on a scale of 0 (none) to 4 (severe). The same scales were used at necropsy on Day 29 to score irritation of the internal vagina (the portion not visible during in-life assessments); any other signs of local or systemic effects were also recorded.

### Necropsy

On Day 29, following external vaginal irritation scoring and ring removal, animals in all Groups were euthanized. At necropsy, a macroscopic examination of the reproductive organs and surrounding tissues was performed and the uterus, cervix, and vagina were collected and fixed in 10% neutral buffered formalin. Microscopic examination of reproductive tissues was conducted routinely processed hematoxylin and eosin-stained slides by a board-certified veterinary pathologist (J.D.V).

Vaginal irritation was scored based on the rabbit vaginal irritation method described by Eckstein et al.¹⁵. For each animal, 3 vaginal regions including the portion adjacent to the cervix (cranial), the middle portion (mid), and the portion at the level of the urethra (uro) were scored separately for 4 parameters (epithelial damage, vascular congestion, edema, and leukocyte infiltration) with each parameter receiving a score of 0 (normal) to 4 (marked).

### Statistical Analyses

Statistical analysis of data was limited to calculation of descriptive statistics, including means, standard deviations (SD), relative standard deviations (RSD), group size for each group and time period (continuous endpoints), and either medians or incident counts for each group and time period (categorical endpoints).

### REFERENCES

1. Mac Bride M B, Rhodes D J, Shuster L T 2010. Vulvovaginal Atrophy. Mayo Clinic Proceedings 85(1):87-94.

2. Freedman R R 2001. Physiology of hot flashes. American Journal of Human Biology 13(4):453-464.

3. 2017. The 2017 hormone therapy position statement of The North American Menopause Society. Menopause 24(7):728-753.

4. 2013. Management of symptomatic vulvovaginal atrophy: 2013 position statement of The North American Menopause Society. Menopause 20(9):886-887.

5. 2014. Practice Bulletin No. 141: Management of Menopausal Symptoms. Obstetrics & Gynecology 123(1):202-216.

6. Rosano G M C, Sarais C, Zoncu S, Mercuro G 2000. The relative effects of progesterone and progestins in hormone replacement therapy. Human Reproduction 15(suppl_1):60-73.

7. Canonico M, Oger E, Plu-Bureau G, Conard J, Meyer G, Lévesque H, Trillot N, Barrellier M-T, Wahl D, Emmerich J, Scarabin P-Y 2007. Hormone Therapy and Venous Thromboembolism Among Postmenopausal Women. Circulation 115(7):840-845.

8. Hamada A L, Maruo T, Samoto T, Yoshida S, Nash H, Spitz I M, Johansson E 2003. Estradiol/progesterone-releasing vaginal rings for hormone replacement therapy in postmenopausal women. Gynecological Endocrinology 17(3):247-254.

9. Holt J D S, Cameron D, Dias N, Holding J, Muntendam A, Oostebring F, Dreier P, Rohan L, Nuttall J 2015. The Sheep as a Model of Preclinical Safety and Pharmacokinetic Evaluations of Candidate Microbicides. Antimicrobial Agents and Chemotherapy 59(7):3761-3770.

10. Moss J A, Malone A M, Smith T J, Kennedy S, Nguyen C, Vincent K L, Motamedi M, Baum M M 2013. Pharmacokinetics of a Multipurpose Pod-Intravaginal Ring Simultaneously Delivering Five Drugs in an Ovine Model. Antimicrobial Agents and Chemotherapy 57(8):3994-3997.

11. Restall B 1966. Histological Observations on the Reproductive Tract of the Ewe. Australian Journal of Biological Sciences 19(4):673-686.

12. Adams N R 1976. Pathological changes in the tissues of infertile ewes with clover disease. Journal of Comparative Pathology 86(1):29-35.

13. Kimball A B, Javorsky E, Ron E S, Crowley W, Langer R 2016. A novel approach to administration of peptides in women: Systemic absorption of a GnRH agonist via transvaginal ring delivery system. Journal of Controlled Release 233:19-28.

14. Draize J H, Woodard G, Calvery H O 1944. Methods for the study of irritation and toxicity of substances applied topically to the skin and mucous membranes. Journal of Pharmacology and Experimental Therapeutics 82(3):377-390.

15. Eckstein P, Jackson M C N, Millman N, Sobrero A J 1969. Comparison of vaginal tolerance tests of spericidal preparations in rabbits and monkeys. Journal of Reproduction and Fertility 20(1):85-93.

16. Sjögren L L, Mørch L S, Løkkegaard E 2016. Hormone replacement therapy and the risk of endometrial cancer: A systematic review. Maturitas 91:25-35.

17. Anderson G L, Judd H L, Kaunitz A M, et al. 2003. Effects of estrogen plus progestin on gynecologic cancers and associated diagnostic procedures: The women's health initiative randomized trial. JAMA 290(13):1739-1748.

18. Goodman M P 2012. Are All Estrogens Created Equal? A Review of Oral vs. Transdermal Therapy. Journal of Women's Health 21(2):161-169.

19. Lethaby A, Ayeleke R O, Roberts H 2016. Local oestrogen for vaginal atrophy in postmenopausal women. Cochrane Database of Systematic Reviews (8). Art. No. CD001500. DOI: 10.1002/14651858.CD001500.pub3.

20. Vincent K L, Bourne N, Bell B A, Vargas G, Tan A, Cowan D, Stanberry L R, Rosenthal S L, Motamedi M 2009. High Resolution Imaging of Epithelial Injury in the Sheep Cervicovaginal Tract: A Promising Model for Testing Safety of Candidate Microbicides. Sexually Transmitted Diseases 36(5):312-318.

A number of embodiments of the invention have been described. Nevertheless, it will be understood that various modifications may be made without departing from the spirit and scope of the invention. Accordingly, other embodiments are within the scope of the following claims.

What is claimed is:

1. An ethylene-vinyl-acetate (EVA), intravaginal ring (IVR), wherein the ring contains at least two segments/fibers, wherein one segment contains 17β-estradiol (E₂) or E₂ equivalent, and the second segment contains progesterone (P) or P equivalent, wherein the EVA ring releases about 4 mg to about 8 mg P per day.

2. The EVA ring according to claim 1, wherein the EVA ring releases about 80 μg/day E₂ to about 160 μg/day E₂.

3. The EVA ring according to claim 2, wherein the EVA ring releases about about 160 μg/day E₂.

4. The EVA ring according to claims 1-3, wherein the EVA ring releases about 4 mg or about 8 mg P per day.

5. The EVA ring according to claims 1-4, wherein the ring is about 57 mm in diameter.

6. The EVA ring according to claims 1-5, wherein the ring has a cross-section diameter of about 5 mm.

7. The EVA ring according to claims 1-6, wherein the EVA ring segment/fiber containing E₂ is prepared at a concentration of about 10 w/w in EVA (28% vinyl acetate content).

8. The EVA ring according to claims 1-7, wherein the EVA segment/fiber length containing E₂ is about 15 mm long.

9. The EVA ring according to claims 1-8, wherein the EVA segment/fiber containing P is prepared using EVA (28% vinyl acetate content) with a final drug loading of 27% w/w.

10. The EVA ring according to claims 1-9, wherein the EVA segment/fiber length is about 74.5 mm or about 148.5 mm in length.

11. The EVA ring according to claims 1-10, wherein the IVR rings have a peak E₂ plasma concentration at about 4 hours after insertion for the EVA rings releasing about 4 mg/day P, and about 2 hours after insertion for the EVA rings releasing about 8 mg/day P.

12. The EVA ring according to claims 1-11, wherein the mean E₂ AUC₀₋₆₇₂ₕᵣ value for rings releasing about 4 mg/day P is about 17,400±2,120 pg*hr/mL.

13. The EVA ring according to claims 1-11, wherein the mean E₂ AUC₀₋₆₇₂ₕᵣ value for EVA rings releasing about 8 mg/day P is about 21,000±3,540 pg*hr/mL.

14. The EVA ring according to claims 1-12, wherein the average plasma concentration over the entire dosing interval (C_{AVG}) is about 25.9±3.16 pg/mL for the rings releasing 4 mg/day P.

15. The EVA ring according to claims 1-11 and claim 13, wherein the average plasma concentration over the entire dosing interval (C_{AVG}) is about 31.3±5.26 pg/mL for the rings releasing about 8 mg/day P.

16. The EVA ring according to claim 14, wherein the in vivo release of P is about 55.9±6.8 m/day.

17. The EVA ring according to claim 15, wherein the in vivo release for P is about 67.3±11.3 μg/day.

18. The EVA ring according to claim 16, wherein the Cₘₐₓ value for P is about 1,590±272 pg/mL.

19. The EVA ring according to claim 17, wherein the Cₘₐₓ value for P is about 2,400±322 pg/mL.

20. The EVA ring according to claim 18, wherein the ring has a C_{AVG}, with mean values of about 357±11.2 pg/mL

21. The EVA ring according to claim 19, wherein the ring has a C_{AVG}, with mean values of about 722±94.1 pg/mL.

22. The EVA ring according to claims 1-21, wherein the device releases an E₂ plasma concentration of about 130 pg/ml to about 180 pg/ml.

23. The EVA ring according to claims 1-22, wherein the EVA IVR ring maintain E₂ concentrations at a quantifiable level at day 29 following IVR insertion into a patient/subject.

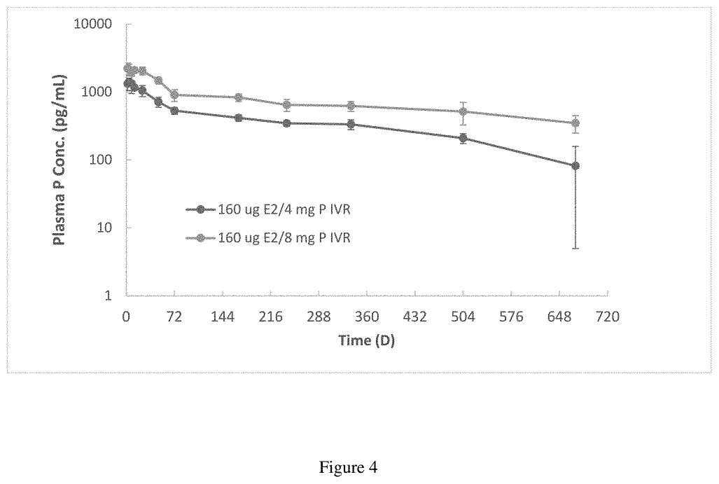

24. An EVA ring according to claim 1, wherein the ring has one of more of the pharmacokinetic properties reported in Table 1-4 and FIGS. 2-4.

25. A method of treating, ameliorating, or preventing vasomotor symptoms associated with perimenopause or menopause in a patient/subject in need of treatment using a ring according to claims 1-24.

26. The method according to claim 25, wherein the symptoms that are treated, ameliorated, or prevented are selected from one or more of hot flashes, flushing, night sweating, mood swings, anxiety, urinary incontinence, cognitive disturbances (memory loss, problems with concentration, arthralgia, weight gain, sexual dysfunction, vaginal disturbances, sleep disturbances, such as insomnia, bone loss, heart disease, atherosclerosis, and heart palpitations.

27. A method of treating, ameliorating, or preventing the symptoms associated with vulvar and vaginal atrophy (VVA) in a patient/subject in need of treatment using a ring according to claims 1-24.

28. A method according to claim 27, wherein the VVA symptoms include, one or more symptoms selected from dryness, burning, itching, vaginal discomfort, vaginal discharge, pain and burning when urinating, urgency with urination, increased urinary tract infections, urinary incontinence, dyspareunia, discomfort with intercourse, decreased vaginal lubrication during sexual activity, shortening and tightening of the vaginal canal, and spotting during intercourse.
